(19) United States
(12) Patent Application Publication
(10) Pub. No.: US 2024/0382533 A1
(43) Pub. Date: Nov. 21, 2024

(54) USE OF HUMAN UMBILICAL CORD MESENCHYMAL STEM CELL-DERIVED SMALL EXTRACELLULAR VESICLE (hucMSC-sEV) IN PREPARATION OF DRUG FOR TREATING TYPE 2 DIABETES MELLITUS (T2DM)

(71) Applicant: JIANGSU UNIVERSITY, Zhenjiang (CN)

(72) Inventors: Hui QIAN, Zhenjiang (CN); Yuntong SUN, Zhenjiang (CN); Fengtian SUN, Zhenjiang (CN); Wenrong XU, Zhenjiang (CN); Cheng JI, Zhenjiang (CN); Hui SHI, Zhenjiang (CN); Zixuan SUN, Zhenjiang (CN)

(21) Appl. No.: 18/600,647

(22) Filed: Mar. 9, 2024

Related U.S. Application Data
(63) Continuation of application No. PCT/CN2023/107208, filed on Jul. 13, 2023.

(30) Foreign Application Priority Data
May 17, 2023 (CN) 202310554417.1

(51) Int. Cl. A61K 35/51; A61P 3/10; C12N 5/0775

(52) U.S. Cl. CPC A61K 35/51; A61P 3/10; C12N 5/0668

(57) ABSTRACT

The present disclosure provides use of a human umbilical cord mesenchymal stem cell-derived small extracellular vesicle (hucMSC-sEV) in preparation of a drug for treating type 2 diabetes mellitus (T2DM), and belongs to the technical field of biomedicine. In the present disclosure, experiments have verified that the hucMSC-SEV can effectively reduce blood glucose in a db/db mouse model, improve a morphological disorder of pancreatic islets, and increase a number of β cells. Moreover, the hucMSC-sEV can also remodel pancreatic β cells by reversing apoptosis and dedifferentiation of the β cells. It is confirmed that the hucMSC-sEV is an effective pharmaceutical ingredient that improves pancreatic islet functions, and can effectively ameliorate T2DM, regulate glucose homeostasis, improve a pancreatic islet structure, and increase a quality of the pancreatic islet β cells, thereby achieving therapeutic effects from a root cause of the disease.

### CROSS REFERENCE TO RELATED APPLICATION

The present application is a continuation of International Patent Application No. PCT/CN2023/107208, filed on Jul. 13, 2023, which claims priority to Chinese Patent Application No. 202310554417.1 filed to the China National Intellectual Property Administration (CNIPA) on May 17, 2023 and entitled “USE OF HUMAN UMBILICAL CORD MESENCHYMAL STEM CELL-DERIVED SMALL EXTRACELLULAR VESICLE (hucMSC-sEV) IN PREPARATION OF DRUG FOR TREATING TYPE 2 DIABETES MELLITUS (T2DM)”, which is incorporated herein by reference in its entirety.

### TECHNICAL FIELD

The present disclosure belongs to the technical field of biomedicine, and specifically relates to use of a human umbilical cord mesenchymal stem cell-derived small extracellular vesicle (hucMSC-sEV) in improving a pancreatic islet function in a type 2 diabetes mellitus (T2DM) model.

### BACKGROUND

Type 2 diabetes mellitus (T2DM) is a chronic metabolic disease with main characteristics of hyperglycemia caused by insulin resistance and relative insulin deficiency. As insulin resistance increases, β cells initially compensate by increasing insulin production and synthesis and increasing β cell proliferation. Long-term insulin resistance and inflammation can lead to β cell stress and compensation, and persistent hyperglycemia and hyperlipidemia can lead to β cell glycolipotoxicity and B cell failure. In turn, β cell dedifferentiation and/or apoptosis occurs, resulting in a decrease in functional B cell mass, leading to the T2DM. The progression of diabetes mellitus involves chronic damage to multiple organs such as blood vessels, kidneys, eyes, and feet. Diabetes mellitus complications are highly disabling and fatal, thus placing a heavy disease burden on patients and families. Therefore, research on effective prevention and treatment of the T2DM is of great benefit.

The current main treatments for T2DM are reasonable diet, moderate exercise, and oral hypoglycemic drugs. Commonly used hypoglycemic drugs mainly enhance the insulin sensitivity of peripheral tissues (such as thiazolidinediones), inhibit hepatic gluconeogenesis (such as biguanides), and increase insulin secretion in a glucose-dependent manner (such as glucagon-like drugs). These drugs are targeted solely at alleviating symptoms by enhancing insulin secretion from remaining β cells and improving insulin sensitivity in T2DM, as well as normalizing blood glucose through tightly controlled exogenous insulin therapy. Although even the secretion function from a small amount of preserved endogenous insulin has been confirmed to have substantial clinical benefits, none of the commonly used antidiabetic therapies targets the maintenance of endogenous B cell mass. Therefore, there is an urgent need to develop novel and safe drugs that can restore β cells and improve β cell functions.

### SUMMARY

In view of this, an objective of the present disclosure is to provide use of a hucMSC-SEV in preparation of a drug for treating T2DM. The present disclosure aims to reduce blood glucose, enhance a function of pancreatic islet β cells, improve a morphological disorder of pancreatic islets, reverse apoptosis and dedifferentiation of β cells, and improve a mass of the pancreatic islet β cells.

To achieve the above objective, the present disclosure provides the following technical solutions:

The present disclosure provides use of a hucMSC-sEV in preparation of a drug for treating T2DM.

Preferably, a preparation method of the hucMSC-SEV includes: culturing a human umbilical cord mesenchymal stem cell (hucMSC) to a cell fusion of 70% to 80%, transferring the hucMSC into a minimum essential medium (MEM)-α medium to allow culturing for 40 h to 50 h, and collecting a resulting medium supernatant to allow differential centrifugation to obtain a precipitate, namely the hucMSC-SEV.

Preferably, the differential centrifugation includes: subjecting the medium supernatant to centrifugation at 250 g to 350 g, 1,500 g to 2,500 g, 9,000 g to 11,000 g, 1,500 g to 2,500 g, and 98,000 g to 120,000 g in sequence.

Preferably, the hucMSC-sEV is injected through a tail vein at a dose of 8 mg/kg to 13 mg/kg.

Preferably, the hucMSC-sEV is used for reducing blood glucose.

Preferably, the hucMSC-sEV is used for improving glucose tolerance.

Preferably, the hucMSC-sEV is used for increasing a volume of pancreatic islets and increase a number of β cells.

Preferably, the hucMSC-sEV is used for reversing apoptosis and dedifferentiation of β cells.

Preferably, the drug further includes a pharmaceutically acceptable carrier.

Compared with the prior art, the present disclosure has the following beneficial effects:

The present disclosure provides novel use of hucMSC-sEV for the treatment of T2DM. Compared with conventional cell therapy, the hucMSC-sEV, as a non-cell therapy, has simple materials, convenient operation, and low immunogenicity. This non-cell therapy can continuously and effectively lower blood glucose, while increasing the mass of pancreatic β cells and greatly reducing side effects. Accordingly, the hucMSC-sEV is conducive to clinical promotion and application, thus providing a novel strategy for the treatment of patients with chronic diseases.

In the present disclosure, experiments have verified that hucMSC-SEV can effectively reduce blood glucose and reduce the weight of mice in a db/db mouse model. Furthermore, H&E staining, Western Blot, immunofluorescence detection, and qRT-PCR staining methods have verified that the hucMSC-sEV can effectively reverse the apoptosis and dedifferentiation of mouse pancreatic islet β cells in T2DM, thereby increasing the number of β cells and improving pancreatic islet functions.

### BRIEF DESCRIPTION OF THE DRAWINGS

FIG. 1 shows an exosome appearance observed by transmission electron microscopy (TEM) of hucMSC-sEV in Example 1;

FIG. 2 shows the particle size detection results of nanoparticle tracking analysis (NTA) of the hucMSC-SEV in Example 1;

FIG. 3 shows the expression of surface characteristic markers of the hucMSC-sEV in the examples detected by Western blot (WB);

FIG. 4 shows the blood glucose values of mice in each group in Example 2;

FIG. 5 shows the body weights of mice in each group in Example 2;

FIG. 6 shows the blood glucose values of mice in each group in Example 2 detected by intraperitoneal glucose tolerance test (IPGTT);

FIG. 7 shows the fasting insulin levels of mice in each group in Example 2 detected by homeostasis model assessment of β-cell function (HOMA-β);

FIG. 8 shows the in vivo imaging of mice in each group in Example 2;

FIG. 9 shows the hematoxylin and eosin (H&E) staining results of pancreatic tissue of mice in each group in Example 2;

FIG. 10 shows a volume of the pancreas of mice in each group in Example 2 detected by tissue immunofluorescence;

FIG. 11 shows the expression of mature B-cell transcription factors Pdx1, FoxO1, Oct4, and Nanog of mice in each group in Example 2 detected by tissue immunofluorescence;

FIG. 12 shows the expression of molecular markers PCNA, Bcl-2, Bax, and cleaved capase3 in the pancreatic tissues of mice in each group in Example 2 detected by multi-strip Western Blot;

FIG. 13 shows the expression of mature B-cell markers Pdx1, FoxO1, and Nkx6.1 in the pancreatic tissue of mice in each group in Example 2 detected by qRT-PCR;

FIG. 14 shows the expression levels of molecular markers PCNA, Bcl-2, and Bax in cells of each group in Example 3 detected by Western Blot;

FIG. 15 shows the expression levels of molecular markers Pdx1 and FoxO1 in cells of each group in Example 3 detected by Western Blot;

FIG. 16 shows the expression levels of molecular markers Oct4 and Nanog in cells of each group in Example 3 detected by Western Blot; and

FIG. 17 shows the expression levels of molecular markers PCNA, Bcl-2, Bax, Pdx1, and FoxO1 in cells of each group in Example 3 detected by cellular immunofluorescence.

### DETAILED DESCRIPTION OF THE EMBODIMENTS

The present disclosure provides use of a hucMSC-sEV in preparation of a drug for treating T2DM.

In the present disclosure, a preparation method of the hucMSC-sEV includes: culturing a hucMSC to a cell fusion of 70% to 80%, transferring the hucMSC into an MEM-α medium to allow culturing for 40 h to 50 h, and collecting a resulting medium supernatant to allow differential centrifugation to obtain a precipitate, namely the hucMSC-sEV. The culturing of the MEM-α medium is conducted for more preferably 48 h. The differential centrifugation preferably includes: subjecting the medium supernatant to centrifugation at 250 g to 350 g, 1,500 g to 2,500 g, 9,000 g to 11,000 g, 1,500 g to 2,500 g, and 98,000 g to 120,000 g for 8 min to 12 min, 15 min to 25 min, 25 min to 35 min, 25 min to 35 min, and 25 min to 35 min in sequence; more preferably at 300 g, 2,000 g, 10,000 g, 2,000 g, and 100,000 g for more preferably 10 min, 20 min, 30 min, 30 min, and 30 min in sequence; and the centrifugation is conducted at preferably 2° C. to 6° C., more preferably 4° C.

In the present disclosure, the hucMSC-sEV is preferably injected through a tail vein; an injection dose is preferably 8 mg/kg to 13 mg/kg, more preferably 10 mg/kg; the hucMSC-sEV can reduce blood glucose; the hucMSC-sEV can improve glucose tolerance; and the hucMSC-sEV can increase the size of pancreatic islets and increase the number of β cells. Animal experiments are conducted in the present disclosure. By monitoring the blood glucose and body weight of mice in each group, it can be seen that the hucMSC-sEV effectively reduces the blood glucose and body weight of db/db mice. By detecting the blood glucose of fasting mice in each group via IPGTT, it is found that the hucMSC-sEV effectively improves the glucose tolerance of db/db mice. Detecting the fasting insulin levels of fasting mice in each group by HOMA-β shows that the function of pancreatic islet β cells in the hucMSC-sEV mice is significantly improved. Detection of pancreatic islets of mice in each group by H&E staining shows that the hucMSC-sEV can significantly increase the number of pancreatic islets, increase the volume of pancreatic islets, and improve the structural disorder of pancreatic islets. Through tissue immunofluorescence detection of pancreatic tissue sections of mice in each group, it is found that the hucMSC-sEV effectively increases β cells and reverses β cell dedifferentiation. Through tissue protein extraction and Western Blot detection of pancreatic tissues of mice in each group, it is found that the hucMSC-sEV effectively reverses the apoptosis of mouse pancreatic islet β cells.

In the present disclosure, the hucMSC-sEV can reverse the apoptosis and dedifferentiation of β cells. The present disclosure has conducted cell experiments. Cell protein extraction and Western Blot detection shows that the hucMSC-sEV effectively reverses the apoptosis and dedifferentiation of INS-1 cells in a high-glucose environment. Cell immunofluorescence detection shows that the hucMSC-sEV effectively promotes cell proliferation in a high-glucose environment and reverses apoptosis and dedifferentiation.

In the present disclosure, the drug preferably further includes a pharmaceutically acceptable carrier; an active ingredient is the hucMSC-sEV, and the active ingredient has a content of preferably 0.1% to 99%; the carrier preferably includes a diluent, a stabilizer, or an excipient; and a dosage form of the drug is preferably an injection. The drug can be administered intraperitoneally, subcutaneously, intramuscularly, intravenously, or nasally.

The technical solution provided by the present disclosure will be described in detail below with reference to the examples, but they should not be construed as limiting the claimed scope of the present disclosure.

### Example 1

Extraction of hucMSC-SEV
1. Isolation and Purification of hucMSCs

(1) After obtaining the consent of the mother, fresh umbilical cords were collected from women who delivered by cesarean section with normal development at term to rule out infectious diseases such as AIDS, syphilis, and hepatitis B.

(2) The surface blood was washed with PBS, arteries, veins, and vascular intima were removed, and then the umbilical cords were transferred to a large dish containing MEM-α medium containing 15% fetal bovine serum. The umbilical cords were cut into square pieces with a side length of about 2 mm by hand with scissors, and transferred into a large dish containing MEM-α culture medium to grow adherently.

(3) The umbilical cords were cultured in a carbon dioxide incubator at 37° C. and 5% CO₂, and the medium was changed every 3 d. After 10 d, when primary cells with fibrous shape and high degree of fusion appeared around the meat piece, the primary cells were subjected to digestion and passage, the third generation cells were selected for experiments.

(4) The mesenchymal stem cells (MSCs) were cultured in a carbon dioxide incubator at 37° C. and 5% CO₂; when the cells were fused to 70% to 80%, the supernatant was discarded. The MSCs were washed twice with PBS and transferred to a specific MEM-α medium to allow culture for 48 h until the cells were completely fused, and a medium supernatant was collected.

2. Isolation of hucMSC-sEV

(1) The collected supernatant was centrifuged at 300×g for 10 min to remove intact cells.

(2) Centrifugation was conducted at 2,000×g for 20 min to further remove dead cells and cell debris.

(3) A resulting upper liquid was transferred to a 10,000 g centrifuge tube to balance, and centrifuged at 10,000 g for 30 min at 4° C. to remove impurity fragments such as organelles.

(4) An upper liquid was transferred to a 100 kD ultrafiltration tube, centrifuged at 4° C. and 2,000 g for 30 min, a supernatant was concentrated, and a bottom concentrated liquid was absorbed, and repeatedly concentrated until there was no residual liquid in the upper layer.

(5) A concentrated supernatant was ultracentrifuged at 4° C. and 100,000 g, an upper liquid was discarded, and an appropriate amount of PBS was added to dissolve the bottom precipitate. After sterilization with a 0.22 μm filter membrane, a protein concentration of the hucMSC-SEV was detected by the BCA method, and stored at −80° C.

3. Identification of hucMSC-sEV

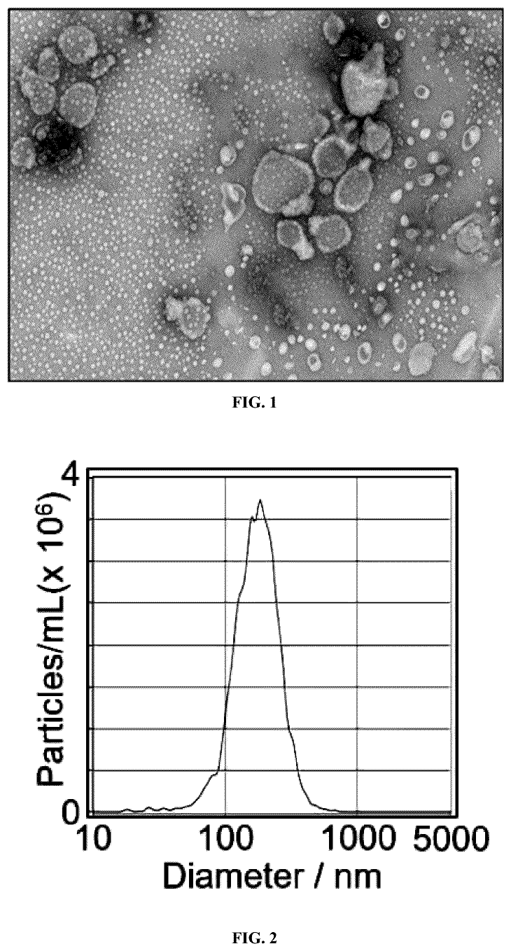

The extracted hucMSC-sEV was observed with a scanning electron microscope (TEM). The results were shown in FIG. 1, the extracted hucMSC-sEV was in a typical vesicle shape.

The particle size was detected by NTA. The results were shown in FIG. 2, the hucMSC-sEV had a particle size of about 150 nm.

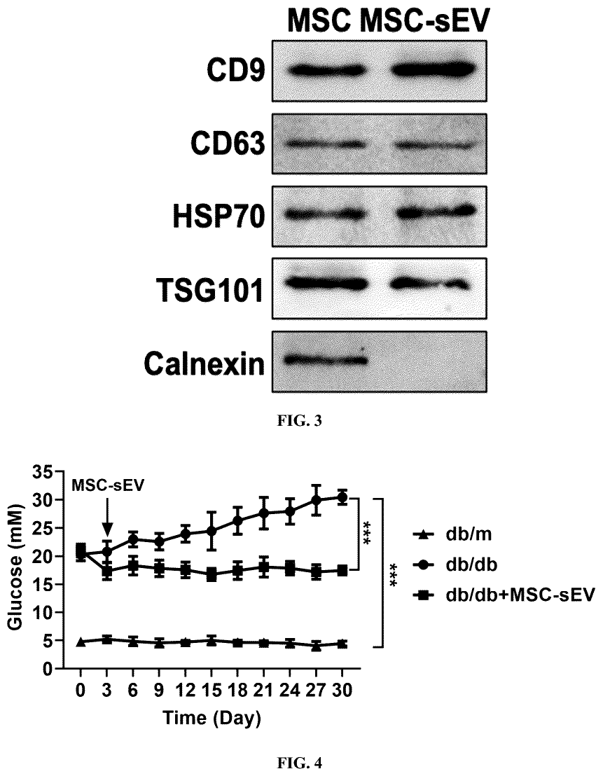

The surface characteristic markers of hucMSC-sEV were detected by WB. The results were shown in FIG. 3, the extracted hucMSC-sEV expressed surface characteristic markers such as CD9, CD63, and TSG101, but did not express calnexin.

It was seen from FIG. 1 to FIG. 3 that the hucMSC-sEV extracted in the present disclosure conformed to the biological characteristics of small extracellular vesicles (sEVs).

### Example 2

Use of hucMSC-sEV in db/db Mouse Model
1. Construction of db/db T2DM Mouse Model

The db/db mouse model was constructed and administered as follows:

15 4-week-old db/m and db/db mice were randomly divided into three groups: (1) db/m group, (2) db/db group, (3) db/db group+Example 1 hucMSC-sEV group, with 5 mice in each group, and then fed normally until they were 8 weeks old. After db/db mice spontaneously developed hyperglycemia, the mice in treatment group were injected with hucMSC-sEV (10 mg/kg/3 d) through the tail vein: the mice were sacrificed 4 weeks later to allow in vivo histological analysis.

### 2. Effect Identification

Monitoring methods of blood glucose and body weight: the blood glucose and body weight of mice were monitored during the 8-12 week treatment, and the mice were fasted for 12 h the day before measurement. The next morning, blood was collected from the tail vein and tested with a Roche blood glucose meter. The results were shown in FIG. 4 and FIG. 5, indicating that the hucMSC-sEV effectively reduced the blood glucose and body weight of db/db mice.

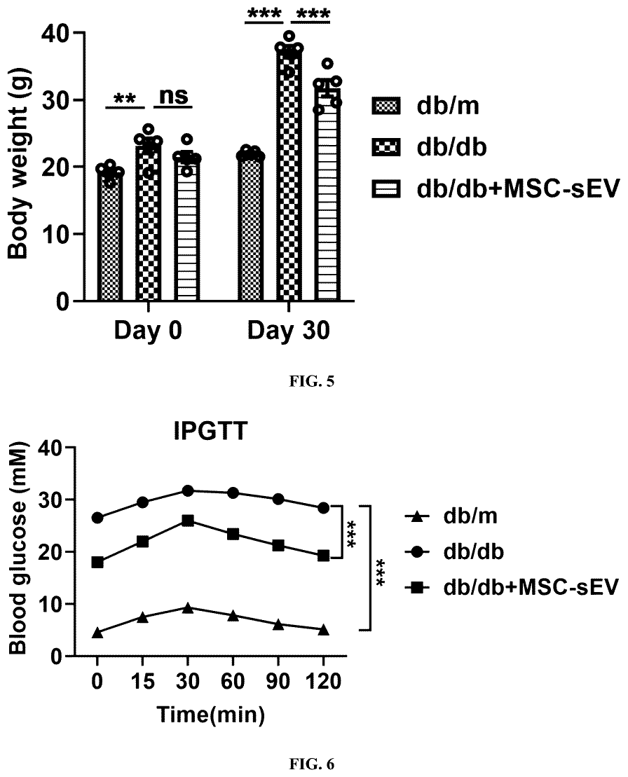

IPGTT detection method: after the mice were fasted for 16 h, glucose (2 g/kg bw) was intraperitoneally injected, and blood glucose was measured at 0, 15, 30, 60, 90, and 120 min after injection. The results were shown in FIG. 6, indicating that the hucMSC-sEV effectively improved the glucose tolerance of db/db mice.

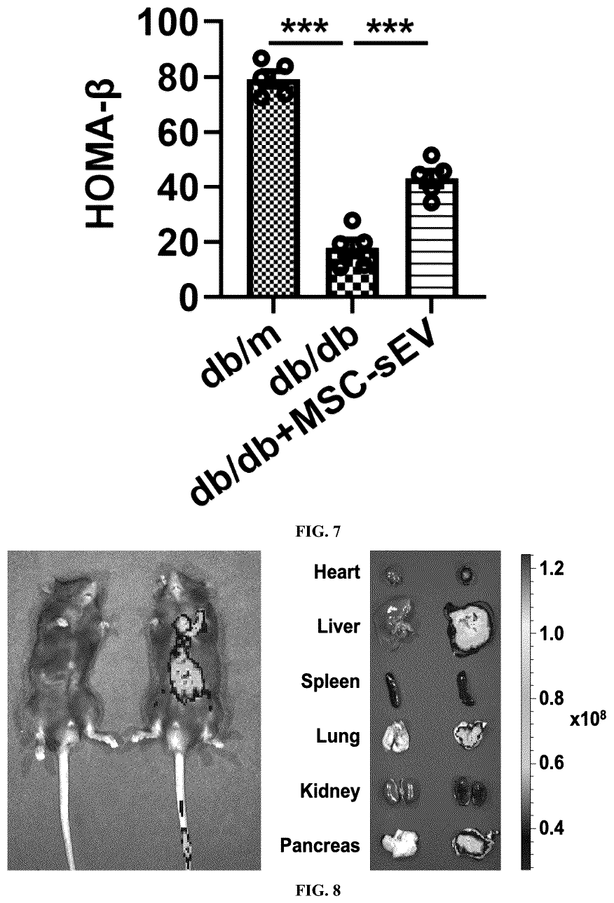

HOMA-β detection method: the mice were fasted for 12 h the day before the measurement, and the fasting blood glucose and fasting insulin levels of the mice were detected. A calculation formula was as follows: HOMA-β=[20× fasting insulin level (mU/1)]/[fasting blood glucose (mmol/l)-3.5]. The results were shown in FIG. 7, indicating that the pancreatic β cell function of mice was significantly improved after the hucMSC-SEV treatment.

Small animal in vivo imaging detection method: DIR dye-labeled hucMSC-sEV is injected through the tail vein, and the labeled hucMSC-sEV was traced using a small animal in vivo imaging instrument. The results were shown in FIG. 8, indicating that the hucMSC-sEV could be taken up by major organs and was abundantly expressed in liver, pancreas, and spleen tissues.

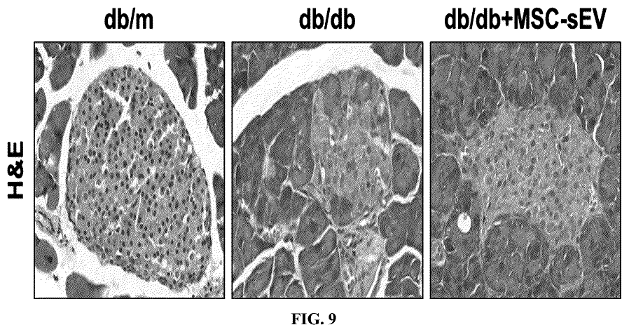

H&E staining detection method: the isolated pancreatic tissue was fixated in 4% formaldehyde overnight, embedded in paraffin, and prepared into 4 μm sections. The sections were stained with H&E and observed under a microscope (Nikon, Tokyo, Japan). The results were shown in FIG. 9, indicating that the number of pancreatic islets in the pancreatic tissue of mice in the hucMSC-sEV treatment group significantly increased, resulting in an increase in pancreatic islet volume and an improvement in pancreatic islet structural disorder.

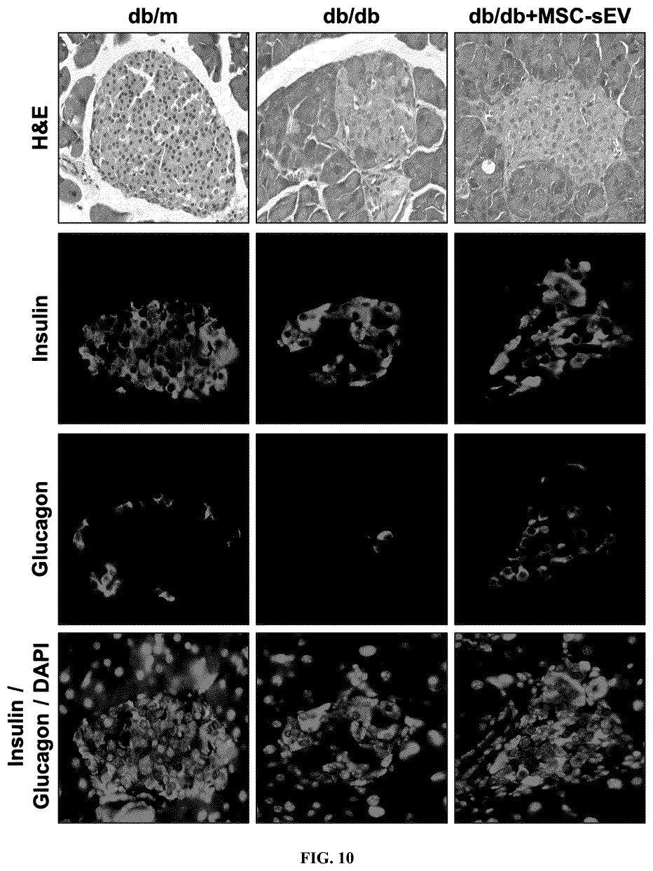

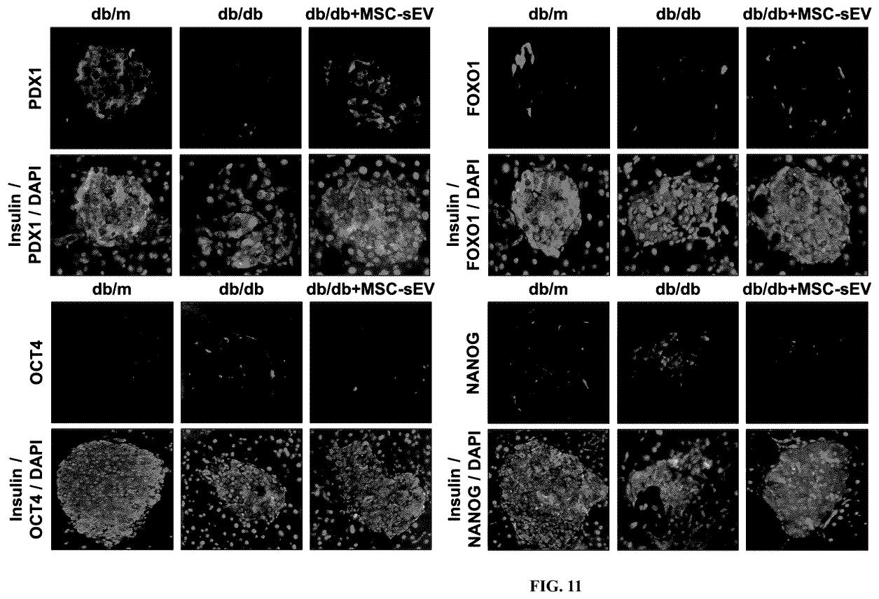

Tissue immunofluorescence detection: 4 μm thick paraffin-embedded mouse pancreatic tissue sections were baked in a 70° C. oven for 2 h, dewaxed in water, placed in 0.01 M citrate buffer, and boiled in a steam pot for 30 min, allowed to cool naturally, blocked with 5% bovine serum albumin (BSA) for 1 h, incubated with specific primary antibody Insulin overnight at 4° C., and then incubated with specific primary antibodies (Glucagon, Pdx1, FoxO1, Oct4, and Nanog) at 4° C. overnight, and then incubated with specific fluorescent secondary antibody at 37° C. in the dark for 1 h, and cell nucleus was observed with DAPI. The sections were observed and images were collected using a laser confocal microscope, and the results were shown in FIG. 10 and FIG. 11.

As shown in FIG. 10, the pancreatic islet volume in pancreatic tissue sections of db/db mice decreased, and the number of insulin-positive β cells decreased. After hucMSC-SEV treatment, the pancreatic islet volume increased and the number of insulin-positive β cells significantly increased, indicating that hucMSC-sEV treatment effectively increased β cells.

As shown in FIG. 11, in pancreatic tissue sections of db/db mice, the number of β cells co-expressing mature β-cell transcription factors Pdx1 and FoxO1 decreased, while the number of cells co-expressing pluripotency markers Oct4 and Nanog increased. After hucMSC-SEV treatment, the number of β cells co-expressing Pdx1 and FoxO1 increased, while the number of β cells co-expressing Oct4 and Nanog decreased in mouse pancreatic tissue sections. This indicated that the hucMSC-sEV reversed β-cell dedifferentiation.

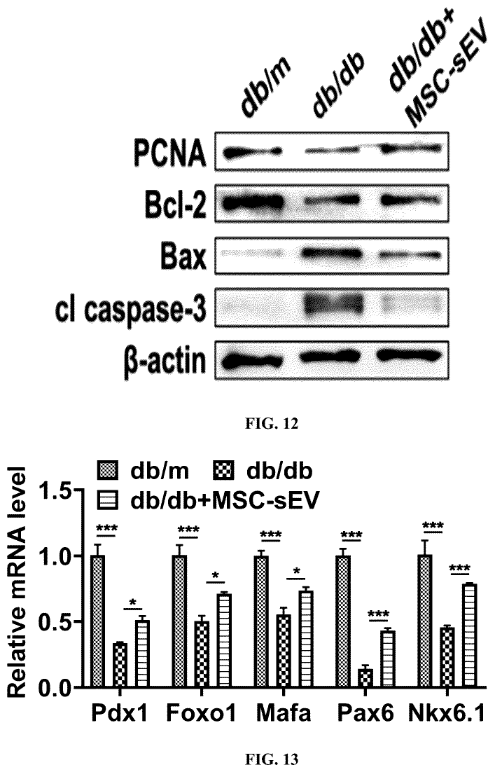

Tissue protein extraction and Western Blot detection methods: 1 cm³ pieces of newly extracted pancreatic tissue were collected, cut into pieces with scissors, washed with PBS and centrifuged at 3,000 g for 5 min to remove blood. The tissue was lysed with RIPA buffer containing phosphatase inhibitors and protease inhibitors, ultrasonically shattered, and then centrifuged at 12,000 g for 10 min, and a supernatant was collected as a total tissue protein. The total tissue protein was added to an equal volume of Loading Buffer and boiled for 10 min, a protein concentration was determined by the BCA method, and 20 μg of protein in each group was separated by polyacrylamide gel electrophoresis. The protein was transferred to a PVDF membrane and blocked with TBST containing 5% skim milk for 60 min, incubated with specific primary antibodies (PCNA, Bcl-2, Bax, and cleaved capase3) overnight at 4° C. separately, and incubated with specific mouse or rabbit secondary antibodies at 37° C. for 1 h. The results were shown in FIG. 12.

As shown in FIG. 12, the expression of proliferation markers such as PCNA and Bcl-2 in the pancreatic tissue of db/db mice was reduced, while the expression of apoptosis markers such as Bax and cleaved capase3 was increased. After hucMSC-sEV treatment, the expression levels of proliferation markers increased and the expression levels of apoptosis markers decreased in mouse pancreatic tissue proteins. This indicated that the hucMSC-sEV effectively reversed mouse pancreatic β-cell apoptosis.

Tissue RNA extraction, reverse transcription, and qRT-PCR detection methods: the pancreatic tissues were cut into pieces with the size of a rice grain, RNA was extracted using Trizol and other organic solvents, a concentration of the RNA was determined with Nanodrop instrument and ND-1000 software, and cDNA was synthesized through Vazyme reverse transcription kit to allow qRT-PCR using the SYBR Green PCR kit. The results were shown in FIG. 13.

As shown in FIG. 13, the expression of mature β-cell markers such as Pdx1, FoxO1, and Nkx6.1 increased significantly in mice in the hucMSC-sEV treatment group.

### Example 3

Use of hucMSC-sEV in Pancreatic Islet Cell Model

Rat islet cell tumor cells (INS-1) were purchased from the Cell Bank of the Chinese Academy of Sciences and cultured in RPMI-1640 (Bioind) medium containing L-glutamine (Thermo Fisher Scientific, USA), 10% fetal bovine serum (FBS, Gibco, Germany), 50 μmol/L β-mercaptoethanol (Procell), and 1% penicillin-streptomycin under standard culture conditions (37° C., 5% CO₂).

The cells were divided into three groups: (1) Control group, (2) HG group: cultured with RPMI-1640 containing 33.3 mmol glucose, and (3) HG+hucMSC-sEV group. Cell treatment method: INS-1 cells were inoculated into a 6-well plate at a density of 2×10⁵, the INS-1 cells were treated with 33.3 mmol high-glucose RPMI-1640 medium for 48 h to simulate a diabetic environment, and hucMSC-sEV (particle number: 1×10⁷/30 μL) was added to treat for 24 h after inducing high glucose injury.

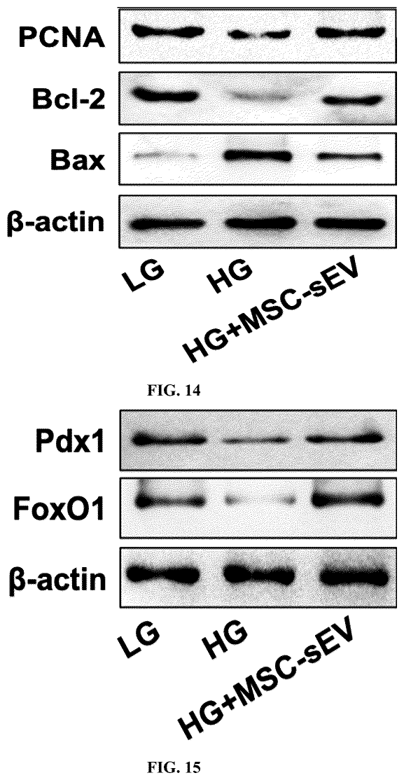

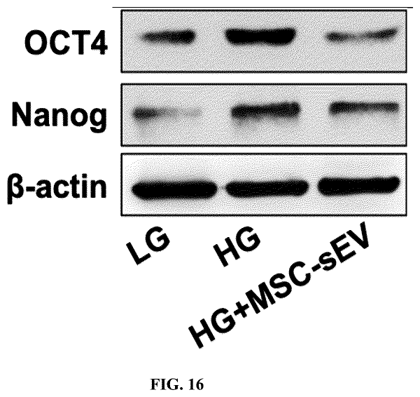

Cell protein extraction and Western Blot detection methods: the cells were lysed using RIPA buffer containing phosphatase inhibitors and protease inhibitors, mixed with a vortex shaker for 5 min, allowed to stand on ice for 10 min, circulated 5 times, and then centrifuged at 12,000 g for 10 min, and a supernatant was collected to obtain a total cell protein. The total cell protein was added to an equal volume of Loading Buffer, and a protein concentration was measured by the BCA method while Western Blot was conducted the same as that of tissue protein, and the cells were incubated with specific primary antibodies (PCNA, Bcl-2, Bax, Pdx1, FoxO1, Oct4, and Nanog) at 4° C. overnight, and then incubated with specific mouse or rabbit secondary antibodies for 1 h at 37° C. The specific results were shown in FIG. 14, FIG. 15, and FIG. 16.

As shown in FIG. 14, after hucMSC-sEV treatment, the expression of PCNA and Bcl-2 in INS-1 cells increased under high glucose environment, the cell proliferation ability was enhanced, the expression of Bax and others was reduced, and apoptosis was weakened. As shown in FIG. 15, the expression of β-cell maturation markers Pdx1 and FoxO1 increased. As shown in FIG. 16, the expression of dedifferentiation markers Oct4 and Nanog decreased, indicating that the hucMSC-sEV effectively reversed the apoptosis and dedifferentiation of INS-1 cells in high-glucose environment.

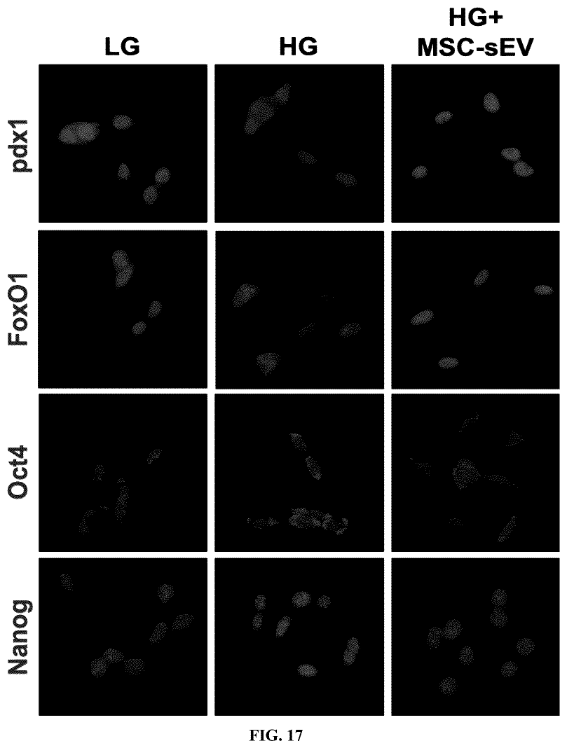

Cell immunofluorescence detection method: the same number of cells was inoculated in a 12-well plate with cell slides placed, and each group was given the different treatments mentioned above. After the cells had grown for 72 h, the cells were removed and spread onto slides, washed 3 times with PBS, fixated in paraformaldehyde for 30 min, ruptured with 0.1% Triton-X100 for 10 min, and blocked with BSA for 30 min. The cells were incubated overnight at 4° C. with specific primary antibodies (PCNA, Bcl-2, Bax, Pdx1, FoxO1, Oct4, and Nanog) separately, and then incubated with specific fluorescent secondary antibodies at 37° C. for 1 h, and the cell nuclei were observed with Hoechst 33,342. The results were shown in FIG. 17.

As shown in FIG. 17, after hucMSC-sEV treatment, the expression of PCNA and Bcl-2 increased, the expression of Bax decreased, and the expression of Pdx1 and FoxO1 increased in INS-1 cells. This indicated that the hucMSC-sEV effectively promoted cell proliferation in a high-glucose environment and reversed apoptosis and dedifferentiation.

In summary, the hucMSC-sEV provided by the present disclosure can reduce blood glucose in the db/db mouse model, enhance the function of pancreatic islet β cells, improve the morphological disorder of pancreatic islets, reverse the apoptosis and dedifferentiation of β cells, and improve the mass of pancreatic β cells.

The above descriptions are merely preferred implementations of the present disclosure. It should be noted that a person of ordinary skill in the art may further make several improvements and modifications without departing from the principle of the present disclosure, but such improvements and modifications should be deemed as falling within the protection scope of the present disclosure.

What is claimed is:

1. A treatment method of type 2 diabetes mellitus (T2DM), comprising treating the T2DM using a human umbilical cord mesenchymal stem cell-derived small extracellular vesicle (hucMSC-sEV).

2. The treatment method according to claim 1, wherein a preparation method of the hucMSC-sEV comprises: culturing a human umbilical cord mesenchymal stem cell (hucMSC) to a cell fusion of 70% to 80%, transferring the hucMSC into a minimum essential medium (MEM)-α medium to allow culturing for 40 h to 50 h, and collecting a resulting medium supernatant to allow differential centrifugation to obtain a precipitate, namely the hucMSC-SEV.

3. The treatment method according to claim 2, wherein the differential centrifugation comprises: subjecting the medium supernatant to centrifugation at 250 g to 350 g, 1,500 g to 2,500 g, 9,000 g to 11,000 g, 1,500 g to 2,500 g, and 98,000 g to 120,000 g in sequence.

4. The treatment method according to claim 1, wherein the hucMSC-sEV is injected through a tail vein at a dose of 8 mg/kg to 13 mg/kg.

5. The treatment method according to claim 1, wherein the hucMSC-sEV is used for reducing blood glucose.

6. The treatment method according to claim 1, wherein the hucMSC-sEV is used for improving glucose tolerance.

7. The treatment method according to claim 1, wherein the hucMSC-sEV is used for increasing a volume of pancreatic islets and increase a number of β cells.

8. The treatment method according to claim 1, wherein the hucMSC-sEV is used for reversing apoptosis and dedifferentiation of β cells.
